(19) United States
(12) Patent Application Publication
(10) Pub. No.: US 2026/0287526 A1
(43) Pub. Date: Sep. 24, 2026

(54) DEVICE AND METHOD FOR TESTING SURFACE HYDRATION INHIBITION OF DRILLING FLUID

(71) Applicant: Southwest Petroleum University, Chengdu (CN)

(72) Inventors: Danchao HUANG, Chengdu (CN); Xing ZHANG, Chengdu (CN); Shenwen FANG, Chengdu (CN); Jianhua WANG, Chengdu (CN); Baojun LI, Chengdu (CN); Chuang ZHANG, Chengdu (CN); Jian ZHANG, Chengdu (CN); Hong CHEN, Chengdu (CN)

(73) Assignee: Southwest Petroleum University, Chengdu (CN)

(21) Appl. No.: 19/390,369

(22) Filed: Nov. 14, 2025

(30) Foreign Application Priority Data
Mar. 21, 2025 (CN) 2025103424297

(51) Int. Cl. G01N 23/20025; G01N 33/28

(52) U.S. Cl. CPC G01N 23/20025; G01N 33/2823

(57) ABSTRACT

A device for testing surface hydration inhibition of drilling fluid includes a reaction vessel, which is provided with a nylon filter cloth, a first fixing frame, and a second fixing frame inside. An end of the nylon filter cloth is detachably connected to the first fixing frame, and the other end of the nylon filter cloth is detachably connected to the second fixing frame. The first fixing frame is detachably connected to an inner wall of a top portion of the reaction vessel, and the second fixing frame is detachably connected to an inner wall of a bottom portion of the reaction vessel. After testing, the basal spacing of the sodium montmorillonite determined by XRD reflects the surface hydration inhibition performance of the drilling fluid. The device is suitable for testing the surface hydration inhibition of various water-based drilling fluids, providing substantial support for the development of drilling fluid technology.

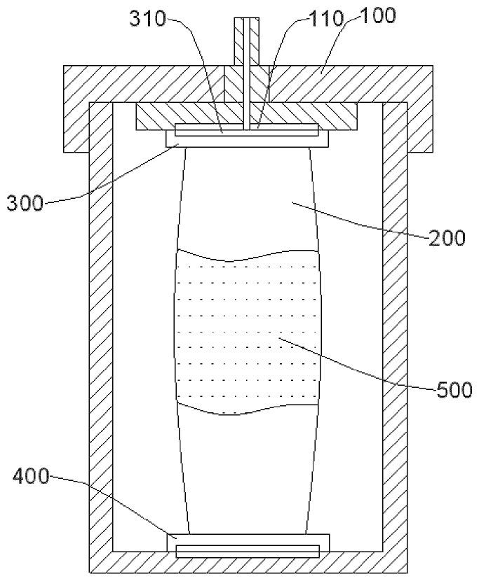

### TECHNICAL FIELD

The disclosure relates to the field of surface hydration inhibition testing technologies, and more particularly to a device and method for testing surface hydration inhibition of drilling fluid.

### BACKGROUND

When drilling fluid interacts with clay minerals, hydration of the clay minerals occurs. For instance, montmorillonite clay minerals have a large number of exchangeable cations in their crystal structures. These cations attract water molecules, leading to volumetric expansion of the clay minerals. Such expansion may cause wellbore instability, resulting in complications including wellbore collapse and diameter reduction, thereby increasing drilling risks and costs.

Currently, the surface hydration inhibition performance of the drilling fluid cannot be effectively evaluated. The primary reason is that surface hydration is too microscopic, requiring the treatment of montmorillonite with the drilling fluid followed by testing of the montmorillonite's microscopic properties (such as basal spacing). The problem lies in the inability to directly introduce sodium montmorillonite into the drilling fluid because the drilling fluid contains other solid components such as bentonite and barite, making it impossible to separate pure sodium montmorillonite afterward. Consequently, analyzing the surface hydration inhibition of the drilling fluid becomes unfeasible.

### SUMMARY

An objective of the disclosure is to provide a device for testing surface hydration inhibition of drilling fluid. This device can simulate a testing environment for the interaction between the drilling fluid and formation clay minerals, thereby providing a fundamental condition for accurately evaluating the surface hydration inhibition of the drilling fluid.

Another objective of the disclosure is to provide a method for using the aforementioned device. This method enables the evaluation of the surface hydration inhibition performance of the drilling fluid by facilitating surface hydration formation on sodium montmorillonite. Different evaluation criteria clearly define the degree of the surface hydration inhibition of the drilling fluid, facilitating researchers and engineers in adjusting and optimizing formulations of the drilling fluid based on the test results to meet the requirements of different drilling projects, thereby improving the safety and efficiency of drilling operations. Furthermore, this method possesses a certain degree of universality and is applicable for testing the surface hydration inhibition of various water-based drilling fluids, providing substantial support for the development of drilling fluid technology.

To address the aforementioned technical problems, the technical solutions adopted in the disclosure are as follows.

In one aspect, an embodiment of the disclosure provides a device for testing surface hydration inhibition of drilling fluid. The device includes a reaction vessel (also referred to reaction tank). The reaction vessel is provided with a nylon filter cloth, a first fixing frame, and a second fixing frame inside. An end of the nylon filter cloth is detachably connected to the first fixing frame, and another end of the nylon filter cloth is detachably connected to the second fixing frame. The first fixing frame is detachably connected to an inner wall of a top portion of the reaction vessel, and the second fixing frame is detachably connected to an inner wall of a bottom portion of the reaction vessel.

By arranging the reaction vessel and equipping it with the nylon filter cloth, the first fixing frame, and the second fixing frame, the characteristic of the nylon filter cloth allowing selective permeation of substances is utilized. This enables material exchange or interaction between the sodium montmorillonite inside the nylon filter cloth and the drilling fluid in the reaction vessel under certain conditions, thereby simulating the hydration inhibition situation of the drilling fluid on clay minerals (exemplified by sodium montmorillonite) in the formation under actual working conditions. Structurally, it allows for easy installation and removal of the nylon filter cloth while ensuring it remains in a stable position within the reaction vessel, guaranteeing full contact and reaction between the nylon filter cloth and the drilling fluid during the test. This setup effectively constructs a testing environment that simulates the interaction between drilling fluid and formation clay minerals, providing a fundamental condition for accurately evaluating the surface hydration inhibition of the drilling fluid. The detachable connection method facilitates assembly, cleaning, and maintenance of the device, enhancing its practicality and operability.

The disclosure creatively employs the nylon filter cloth. The nylon filter cloth can achieve a mesh size of up to tens of thousands, with pore sizes reaching the nanoscale. Furthermore, it offers better temperature resistance (nylon material generally has a minimum temperature resistance of 120° C. and can withstand up to 250° C.), solving the problem in the prior art where reactions could only be conducted at room temperature. The disclosure enables the evaluation of inhibition performance under high-temperature and high-pressure conditions.

Since only liquid phase components in the drilling fluid affect the montmorillonite, while solid phases have no effect, and given that the prior art cannot separate pure sodium montmorillonite where excess solid components would interfere with basal spacing measurement, leading to inaccurate test results, the disclosure innovatively uses the nylon filter cloth to isolate the sodium montmorillonite from the solid phases in the drilling fluid. This allows the liquid phase components to enter the nylon filter cloth and interact with the sodium montmorillonite. Consequently, it can realistically recreate the environment of the sodium montmorillonite in the drilling fluid while enabling the separation of the sodium montmorillonite. This addresses the issue of the solid components affecting the test results in subsequent basal spacing measurement, making the results more accurate and reliable while simulating the real environment.

In some embodiments of the disclosure, the nylon filter cloth has a mesh size greater than 80000 mesh (i.e., 0.1 micrometers).

In some embodiments of the disclosure, both the first fixing frame and the second fixing frame are embedded with first magnetic blocks. The inner wall of the top portion and the inner wall of the bottom portion of the reaction vessel are embedded with second magnetic blocks. The first magnetic blocks and the second magnetic blocks have opposite magnetic poles. The mutual attraction between the magnetic poles is utilized to achieve fixed connections between the first fixing frame and the inner wall of the top portion of the reaction vessel, and between the second fixing frame and the inner wall of the bottom portion of the reaction vessel. This magnetic fixation method does not require additional complex connecting components; relying solely on the magnetic force between the magnetic blocks can stably fix the fixing frames to the inner wall of the reaction vessel. Compared to traditional fixation methods, magnetic fixation offers the advantages of simple and quick operation, significantly reducing the assembly time of the device. Simultaneously, as no complex connection structures are used, it reduces the internal space occupation of the device, making the internal space of the reaction vessel more regular, which is beneficial for the layout of the nylon filter cloth inside the reaction vessel and the flow of the drilling fluid. Moreover, magnetic fixation facilitates easy adjustment of the position of the fixing frames according to testing requirements, enhancing the flexibility of the device. Additionally, the magnetic blocks have a long service life and are not easily damaged, reducing the maintenance cost of the device.

In some embodiments of the disclosure, the reaction vessel is an aging vessel. The aging vessel typically possess good sealing performance, resistance to high temperature and high pressure, and certain corrosion resistance. During the testing process of the surface hydration inhibition of the drilling fluid, the drilling fluid and the sodium montmorillonite within the nylon filter cloth need to be subjected to aging treatment under specific temperature and pressure environments to simulate the actual situation of the drilling fluid working underground for extended periods. The aging vessel can withstand the high temperatures and certain pressures that may be generated during the test, prevent drilling fluid leakage, and ensure the stability of the testing environment. The material and structural characteristics of the aging vessel enable it to provide a stable and reliable reaction environment for the test, ensuring the accuracy and reliability of the test results. Its good sealing performance can effectively prevent drilling fluid volatilization or the entry of external impurities into the reaction system, avoiding interference with the test results. The resistance to high temperature and high pressure allows the device to adapt to a wide range of test temperature and pressure conditions, broadening the application scope of the device, which can be used to simulate drilling fluid performance tests under different formation depth conditions. The corrosion resistance extends the service life of the reaction vessel, reduces equipment replacement costs due to vessel corrosion, and improves the overall economy and durability of the device.

In some embodiments of the disclosure, both the first fixing frame and the second fixing frame are provided with clip mechanisms. The two ends of the nylon filter cloth are clamped by the clip mechanisms to achieve detachable fixation.

It should be noted that the aforementioned clip mechanism is a common structure in the prior art, and any structure that fulfills the function of fixing the nylon filter cloth can be used, which is not limited herein.

In another aspect, an embodiment of the disclosure provides a method for testing surface hydration inhibition of drilling fluid using the aforementioned device. The method includes the following steps:
S1. filling the aging vessel with 400 milliliters (mL) of the drilling fluid, and loading sodium montmorillonite into the nylon filter cloth;
S2. fixing two ends of the nylon filter cloth using the first fixing frame and the second fixing frame;
S3. fixing the second fixing frame to the inner wall of the bottom portion of the aging vessel, and fixing the first fixing frame to the inner wall of the top portion of the aging vessel;
S4. raising the temperature of a roller oven to a specified temperature, placing the aging vessel inside the roller oven to perform hot rolling;
S5. after the hot rolling is completed, cooling and opening the aging vessel, taking out the nylon filter cloth, taking out the sodium montmorillonite, and placing the sodium montmorillonite into a centrifuge for centrifugation;
S6. after the centrifugation, pouring out the supernatant liquid and collecting the centrifuged sodium montmorillonite; and
S7. using X-ray diffraction (XRD) to determine basal spacing of the centrifuged sodium montmorillonite, and evaluating the inhibition performance of the drilling fluid based on the basal spacing.

In some embodiments of the disclosure, in S1, the amount of the sodium montmorillonite used is 8 grams (g).

In some embodiments of the disclosure, in S1, the sodium montmorillonite is high-purity sodium montmorillonite with a purity greater than 99%.

In some embodiments of the disclosure, in S4, the specified temperature is in a range of 25° C. to 230° C., the hot rolling time is in a range of 12 hours to 48 hours, and the rotational speed of the roller oven is 50 revolutions per minute (r/min).

In some embodiments of the disclosure, in S4, the specified temperature is in a range of 100° C. to 200° C.

In some embodiments of the disclosure, in S4, time for the hot rolling is in a range of 16 hours to 45 hours.

In some embodiments of the disclosure, in S5, the centrifugation includes centrifugation with a rotational speed of 10000 r/min at 25° C. for 20 minutes.

In some embodiments of the disclosure, in S7, the XRD parameters include a diffraction wavelength λ of 0.154056 nanometers (nm), an operating voltage of 40 kilovolts (kV), a current of 30 milliamperes (mA), and a scanning angle of 2θ from 3° to 40°.

In some embodiments of the disclosure, in S7, the criteria for evaluating the inhibition performance of the drilling fluid includes:
when the basal spacing is less than 1.00 nm and the centrifuged sodium montmorillonite contains zero hydration layers, the surface hydration inhibition is evaluated as no hydration;
when the basal spacing is in a range of 1.00 nm to 1.30 nm and the centrifuged sodium montmorillonite contains one hydration layer, the surface hydration inhibition is evaluated as good inhibition;
when the basal spacing is in a range of 1.30 nm to 1.50 nm and the centrifuged sodium montmorillonite contains two hydration layers, the surface hydration inhibition is evaluated as relatively good inhibition;
when the basal spacing is in a range of 1.50 nm to 1.80 nm and the centrifuged sodium montmorillonite contains three hydration layers, the surface hydration inhibition is evaluated as relatively poor inhibition;
when the basal spacing is in a range of 1.80 nm to 2.00 nm and the centrifuged sodium montmorillonite contains four hydration layers, the surface hydration inhibition is evaluated as poor inhibition; or when the basal spacing is greater than 2.00 nm and the centrifuged sodium montmorillonite contains five or more hydration layers, the surface hydration inhibition is evaluated as no inhibition.

By controlling the amounts of the drilling fluid and the sodium montmorillonite used, as well as the purity of the sodium montmorillonite, standardized starting conditions for the subsequent reaction are provided. The drilling fluid, as the test subject, interacts with the sodium montmorillonite, which is key to evaluating its hydration inhibition. Specific amounts and high-purity sodium montmorillonite ensure consistency and repeatability of the reaction. Accelerating the reaction between the drilling fluid and the sodium montmorillonite by increasing the temperature and extending the duration simulates the actual working condition where the drilling fluid is subjected to high temperatures for extended periods underground, providing sufficient opportunity for components in the drilling fluid to undergo reactions related to hydration inhibition with the sodium montmorillonite. Using centrifugal force to separate substances adsorbed on or combined with the surface of the sodium montmorillonite allows for subsequent accurate measurement of the basal spacing of the sodium montmorillonite. XRD technology, based on the principle of interaction between X-rays and crystalline substances, determines the crystal structure and lattice spacing by measuring the position and intensity of diffraction peaks, thereby reflecting the hydration state of the sodium montmorillonite and further evaluating the inhibition ability of the drilling fluid against its surface hydration. By strictly controlling parameters in each step, such as the amount of the drilling fluid, the purity of the sodium montmorillonite, the reaction temperature and time, the centrifugation conditions, and the XRD testing parameters, the accuracy and reliability of the test results can be improved, providing scientific and effective means for the research and development, quality control, and performance evaluation of the drilling fluid. Different evaluation criteria clearly define the quality degree of the drilling fluid's surface hydration inhibition, facilitating researchers and engineers to adjust and optimize drilling fluid formulations based on the test results to meet the requirements of different drilling projects, improving the safety and efficiency of drilling operations. Simultaneously, the method has a certain universality and is applicable to the surface hydration inhibition test of various types of drilling fluids, providing substantial support for the development of drilling fluid technology.

Compared with the prior art, the embodiments of the disclosure have at least the following advantages or beneficial effects.
1. Since the drilling fluid also contains solid phases (bentonite, barite, plugging materials), if the sodium montmorillonite is directly added, it cannot be retrieved. The disclosure solves the problem of separating the sodium montmorillonite.
2. Inhibition tests in the prior art are all conducted at room temperature. The disclosure solves the problem of evaluating inhibition under high-temperature and high-pressure conditions.
3. In the prior art, evaluation is performed using the filtrate. However, the content of treating agents in the filtrate differs from that in the drilling fluid because the mud cake adsorbs and retains a portion, leading to inaccuracies. The disclosure uses the nylon filter cloth. The nylon filter cloth is soft, and under rolling conditions, no mud cake forms on its surface, thus resulting in greater accuracy.
4. In the prior art, a high-temperature high-pressure fluid loss test is first conducted, and then the filtrate is used to interact with the montmorillonite. In contrast, the disclosure directly uses the drilling fluid to interact with the montmorillonite, eliminating the step of high-temperature high-pressure fluid loss testing, thus simplifying the process.
5. Criteria for inhibition evaluation are established.

### BRIEF DESCRIPTION OF DRAWING

To illustrate the technical solutions in the embodiments of the disclosure more clearly, the following briefly introduces the accompanying drawing required for describing the embodiments. It should be understood that the accompanying drawing below merely shows some embodiments of the disclosure and therefore should not be considered as limiting the scope. For those skilled in the art, other related drawings may be derived from the accompanying drawing without creative efforts.

FIGURE is a schematic structural diagram of a device for testing surface hydration inhibition of drilling fluid according to the disclosure.

Reference Numerals: 100: reaction vessel; 110: second magnetic block; 200: nylon filter cloth; 300: first fixing frame; 310: first magnetic block; 400: second fixing frame; 500: sodium montmorillonite.

### DETAILED DESCRIPTION OF EMBODIMENTS

The objectives, technical solutions, and advantages of the embodiments of the disclosure are described clearly and completely below with reference to the accompanying drawing. Apparently, the described embodiments are only a part, not all, of the embodiments of the disclosure. All other embodiments obtained by those of ordinary skill in the art based on the embodiments herein without creative efforts shall fall within the protection scope of the disclosure.

It should be noted that, in case of conflict, the embodiments in the disclosure and the features therein may be combined with each other. The disclosure will be described in detail below with reference to the accompanying drawing and embodiments.

The following describes the features and performance of the disclosure in further detail with reference to embodiments.

### Embodiment 1

Please refer to FIGURE, FIGURE shows a schematic structural diagram of a device for testing surface hydration inhibition of drilling fluid according to an embodiment of the disclosure.

The disclosure provides a device for testing surface hydration inhibition of drilling fluid. The device includes a reaction vessel 100. A nylon filter cloth 200, a first fixing frame 300, and a second fixing frame 400 are disposed inside the reaction vessel 100. An end of the nylon filter cloth 200 is detachably connected to the first fixing frame 300, and the other end of the nylon filter cloth 200 is detachably connected to the second fixing frame 400. The first fixing frame 300 is detachably connected to an inner wall of a top portion of the reaction vessel 100. The second fixing frame 400 is detachably connected to an inner wall of a bottom portion of the reaction vessel 100.

To achieve the detachable connection between the first fixing frame 300, the second fixing frame 400, and the reaction vessel 100, the first fixing frame 300 and the second fixing frame 400 are respectively embedded with first magnetic blocks 310, and the inner wall of the top portion and the inner wall of the bottom portion of the reaction vessel 100 are respectively embedded with second magnetic blocks 110. The first magnetic block 310 and the second magnetic block 110 have opposite magnetic poles. The mutual attraction between the magnetic poles is utilized to achieve the fixed connections between the first fixing frame 300 and the inner wall of the top portion of the reaction vessel 100, and between the second fixing frame 400 and the inner wall of the bottom portion of the reaction vessel 100. This magnetic fixation method does not require additional complex connecting components. Relying solely on the magnetic force between the magnetic blocks can stably fix the fixing frames to the inner wall of the reaction vessel 100. Compared to traditional fixation methods, magnetic fixation offers the advantages of simple and quick operation, significantly reducing the assembly time of the device. Simultaneously, as no complex connection structures are used, it reduces the internal space occupation of the device, making the internal space of the reaction vessel 100 more regular, which is beneficial for the layout of the nylon filter cloth 200 inside the reaction vessel 100 and the flow of the drilling fluid. Moreover, magnetic fixation facilitates easy adjustment of the position of the fixing frames according to testing requirements, enhancing the flexibility of the device. Additionally, the magnetic blocks have a long service life and are not easily damaged, reducing the maintenance cost of the device.

To further define the type of the reaction vessel 100, the reaction vessel 100 is an aging vessel. The aging vessel typically possess good sealing performance, resistance to high temperature and high pressure, and certain corrosion resistance. During the testing process of the surface hydration inhibition of the drilling fluid, the drilling fluid and the sodium montmorillonite 500 within the nylon filter cloth 200 need to be subjected to aging treatment under specific temperature and pressure environments to simulate the actual situation of the drilling fluid working underground for extended periods. The aging vessel can withstand the high temperatures and certain pressures that may be generated during the test, prevent drilling fluid leakage, and ensure the stability of the testing environment. The material and structural characteristics of the aging vessel enable it to provide a stable and reliable reaction environment for the test, ensuring the accuracy and repeatability of the test results. Its good sealing performance can effectively prevent drilling fluid volatilization or the entry of external impurities into the reaction system, avoiding interference with the test results. The resistance to high temperature and high pressure allows the device to adapt to a wide range of test temperature and pressure conditions, broadening the application scope of the device, which can be used to simulate drilling fluid performance tests under different formation depth conditions. The corrosion resistance extends the service life of the reaction vessel 100, reduces equipment replacement costs due to vessel corrosion, and improves the overall economy and durability of the device.

To achieve connection and fixation of the nylon filter cloth 200, the first fixing frame 300 and the second fixing frame 400 are each provided with a clip mechanism. The clip mechanism enables detachable fixation of two ends of the nylon filter cloth 200.

During use, the aging vessel is opened and filled with the drilling fluid. The sodium montmorillonite 500 is placed into the nylon filter cloth 200. The clip mechanisms are used to connect one end of the nylon filter cloth 200 to the first fixing frame 300 and the other end to the second fixing frame 400. The first fixing frame 300 is attached to the inner wall of the top portion of the aging vessel via the magnetic attraction between the first magnetic block 310 and the second magnetic block 110. The second fixing frame 400 is attached to the inner wall of the bottom portion of the aging vessel via the magnetic attraction between the first magnetic block 310 and the second magnetic block 110. This completes the fixation of the nylon filter cloth 200 within the aging vessel. After the aging vessel is closed, the test can begin.

### Embodiment 2

This embodiment provides a method for testing surface hydration inhibition of drilling fluid using the described device. The method includes the following steps S1 to S7.
S1. The aging vessel is filled with 400 mL of the drilling fluid, and the drilling fluid is a potassium-based polysulfonated drilling fluid system, which includes 2% bentonite+3% sulfonated lignite+3% sulfonated resin+7% potassium chloride+2% sulfonated asphalt+0.2% NaOH+45% barite. 8 g of sodium montmorillonite 500 is loaded into the nylon filter cloth 200.
S2. Two ends of the nylon filter cloth 200 are fixed using the first fixing frame 300 and the second fixing frame 400.
S3. The second fixing frame 400 is fixed to the inner wall of the bottom portion of the aging vessel, and the first fixing frame 300 is fixed to the inner wall of the top portion of the aging vessel.
S4. The temperature of a roller oven is raised to 150° C., and the aging vessel is placed inside the roller oven to perform hot rolling for 16 hours. The rotational speed of the roller oven is 50 r/min.
S5. After the hot rolling is completed, the aging vessel is cooled and opened. The nylon filter cloth 200 is taken out, and the sodium montmorillonite 500 is collected and placed into a centrifuge for centrifugation at 10000 r/min and 25° C. for 20 minutes.
S6. After the centrifugation, the supernatant liquid is poured out and the centrifuged sodium montmorillonite 500 is collected.
S7. The basal spacing of the centrifuged sodium montmorillonite 500 is determined by XRD under the following conditions: the diffraction wavelength λ=0.154056 nm, the operating voltage of 40 kV, the current of 30 mA, the scanning angle 2θ=3° to 40°. The inhibition performance of the drilling fluid is evaluated based on the basal spacing. The evaluation method is shown in Table 1 below.

TABLE 1

| | | Surface Hydration |
|---|---|---|
| | | Inhibition |
| Basal Spacing | Hydration Layers | Evaluation |
| Less than | Dry montmorillonite | No hydration |
| 1.00 nm | | |
| 1.00 nm to | Montmorillonite with | Good inhibition |
| 1.30 nm | 1 hydration layer | |
| 1.30 nm to | Montmorillonite with | Relatively good |
| 1.50 nm | 2 hydration layers | inhibition |
| 1.50 nm to | Montmorillonite with | Relatively poor |
| 1.80 nm | 3 hydration layers | inhibition |
| 1.80 nm to | Montmorillonite with | Poor inhibition |
| 2.00 nm | 4 hydration layers | |
| Greater than | Montmorillonite with | No inhibition |
| 2.00 nm | 5 or more hydration layers | |

In this embodiment, the basal spacing of the sodium montmorillonite 500 determined by XRD is 1.25 nm. Referring to Table 1, the inhibition performance of the potassium-based polysulfonated drilling fluid system is evaluated as good.

### Comparative Example 1

This comparative example uses a conventional method from the prior art to evaluate the inhibition of the potassium-based polysulfonated drilling fluid system. The specific steps are as follows.
(1) Core preparation: the bentonite is dried in an oven at 105° C. (+3° C.) for 4 hours. Then 10.0 g of the dried bentonite is weighed and loaded into a measuring cylinder of a shale linear swell meter. The cylinder is placed in a press and subjected to a pressure of 4.0 MPa for 5 minutes to form the core required for linear expansion rate measurement. The height of the core, AL, is recorded.
(2) Sample solution preparation: three separate solutions are prepared by adding 3.00 g of different inhibitors to 300 mL of distilled water each. These solutions are then stirred for 20 minutes for complete dissolution.
(3) After the measuring cylinder containing the core is installed on the shale linear swell meter, each sample solution is added to the cylinder, and the initial reading Rₒ and the reading Rₓ after different time intervals are recorded. Simultaneously, a blank test is carried out using distilled water.

The linear expansion rates of different inhibitors are evaluated according to the standard “SY/T6335-1997”. The calculation formula for the linear expansion rate is as follows:

$${s}_{r} = \frac{Δ R}{Δ L} \times 100 \% , \qquad Formula 1$$

where Sᵣ is the linear expansion rate of the bentonite, ΔR is the expansion amount of the bentonite, obtained from (Rₓ−Rₒ), and ΔL is the core height, mm.

According to Formula 1, Sᵣ of the potassium-based polysulfonated drilling fluid system is 12%, and the inhibition is evaluated as good. Based on the results, the evaluation result in the comparative example 1 is consistent with that in the embodiment 2, proving the feasibility and accuracy of the evaluation method in the embodiment 2.

### Comparative Example 2

This comparative example is basically consistent with the embodiment 2, except that the sodium montmorillonite 500 is not placed in the nylon filter cloth 200 but is directly placed into the aging vessel to contact the drilling fluid system. After the test, the basal spacing determined by XRD is 1.67 nm, and the inhibition is evaluated as relatively poor. The results show that the result of the comparative example 2 is inconsistent with those of the embodiment 2 and comparative example 1. This is because when the sodium montmorillonite 500 interacts with the drilling fluid, the solid phase components in the drilling fluid also contact the sodium montmorillonite 500. Although the solid phase components do not affect surface hydration, the solid phase components remaining on the sodium montmorillonite 500 during XRD basal spacing measurement interfere with the results, causing inaccuracies. Separating the solid phase components from the sodium montmorillonite 500 is difficult and often incomplete. Therefore, even if separation is attempted, the obtained basal spacing measurement results are similarly inaccurate, leading to incorrect evaluation results.

### Embodiment 3

This embodiment provides a method for testing surface hydration inhibition of drilling fluid using the described device. The method includes the following steps S1 to S7.
S1. The aging vessel is filled with 400 mL of the drilling fluid, and the drilling fluid is a polymer drilling fluid system, which includes 3% bentonite+0.2% anionic polyacrylamide+0.3% polyanionic cellulose+0.1% xanthan gum+0.5% polyamine inhibitor+30% barite. 8 g of sodium montmorillonite 500 is loaded into the nylon filter cloth 200.
S2. Two ends of the nylon filter cloth 200 are fixed using the first fixing frame 300 and the second fixing frame 400.
S3. The second fixing frame 400 is fixed to the inner wall of the bottom portion of the aging vessel, and the first fixing frame 300 is fixed to the inner wall of the top portion of the aging vessel.
S4. The temperature of a roller oven is raised to 100° C., and the aging vessel is placed inside the roller oven to perform hot rolling for 16 hours. The rotational speed of the roller oven is 50 r/min.
S5. After the hot rolling is completed, the aging vessel is cooled and opened. The nylon filter cloth 200 is taken out, then the sodium montmorillonite 500 is collected and placed into a centrifuge for centrifugation at 10000 r/min and 25° C. for 20 minutes.
S6. After the centrifugation, the supernatant liquid is poured out and the centrifuged sodium montmorillonite 500 is collected.
S7. The basal spacing of the centrifuged sodium montmorillonite 500 is determined by XRD under the following conditions: the diffraction wavelength λ=0.154056 nm, the operating voltage of 40 kV, the current of 30 mA, the scanning angle 2θ=3° to 40°. The inhibition performance of the drilling fluid is evaluated based on the basal spacing. The evaluation method is shown in Table 1 below.

TABLE 1

| | | Surface Hydration |
|---|---|---|
| | | Inhibition |
| Basal Spacing | Hydration Layers | Evaluation |
| Less than | Dry montmorillonite | No hydration |
| 1.00 nm | | |
| 1.00 nm to | Montmorillonite with | Good inhibition |
| 1.30 nm | 1 hydration layer | |
| 1.30 nm to | Montmorillonite with | Relatively good |
| 1.50 nm | 2 hydration layers | inhibition |
| 1.50 nm to | Montmorillonite with | Relatively poor |
| 1.80 nm | 3 hydration layers | inhibition |
| 1.80 nm to | Montmorillonite with | Poor inhibition |
| 2.00 nm | 4 hydration layers | |
| Greater than | Montmorillonite with | No inhibition |
| 2.00 nm | 5 or more hydration layers | |

In this embodiment, the basal spacing of the sodium montmorillonite 500 determined by XRD is 1.55 nm. Referring to Table 1, the surface hydration inhibition of the polymer drilling fluid system is evaluated as relatively poor.

### Comparative Example 3

This comparative example uses a conventional method from the prior art, specifically the rolling recovery test, to evaluate the inhibition of the polymer drilling fluid system.

In this test, the rolling recovery rate of the polymer drilling fluid system for shale (120° C., 16 h) is used as the evaluation indicator. The specific test steps are as follows.
(1) The collected drill cuttings are sieved using a double-deck sieve with sieve opening side lengths of 3.2 mm and 2.0 mm, respectively. The cuttings that pass through the 3.2 mm sieve are collected.
(2) 50.0 g (accurate to 0.1 g) of the prepared drill cuttings are loaded into a high-temperature cell (also referred to high-temperature tank) containing 350 mL of the evaluation fluid and the lid is tightened.
(3) The loaded high-temperature cell is placed into a drilling fluid roller oven at 120° C.+3° C. and rolled for 16 hours.
(4) After constant temperature rolling for 16 hours, the high-temperature cell is taken out and cooled to room temperature. All the liquid and rock samples are poured from the cell onto a sieve with a sieve opening side length of 0.42 mm. Wet sieve wash is carried out on the liquid and rock samples in a trough containing tap water for 1.0 minute.
(5) The residual rock sample is placed into a blast oven and dried at 105° C.±3° C. for 4 h. After removal and cooling, it is allowed to stand in air for 24 h. Subsequently, the sample is weighed to the nearest 0.1 g to calculate the primary rolling recovery rate (R).

The rolling recovery rate R obtained from the above steps is 95%, and the inhibition is evaluated as good. The results show that the evaluation result of the comparative example 3 is inconsistent with that of the embodiment 3.

### Comparative Example 4

This comparative example is basically the same as the comparative example 1, except that the drilling fluid system is the polymer drilling fluid system. According to Formula 1, the Sy of the polymer drilling fluid system is 31%, indicating relatively poor inhibition. This result is consistent with the result in the embodiment 3 but inconsistent with the rolling recovery experimental result in the comparative example 3. This is because the clay hydration process involves crystal layer expansion after water absorption. When expansion reaches a certain degree, dispersion occurs. The rolling recovery rate primarily evaluates the dispersion ability. However, when the clay absorbs water and expands but has not yet dispersed, the rolling recovery rate would indicate good inhibition, which is clearly inconsistent with the facts. In practical use, this drilling fluid system can hardly achieve the technical effect evaluated in the comparative example 3. Therefore, combining the embodiment 3, comparative example 3, and comparative example 4, it can be concluded that the evaluation result of the rolling recovery test is inaccurate.

### Embodiment 4

This embodiment provides a method for testing surface hydration inhibition of drilling fluid using the described device. The method includes the following steps S1 to S7.
S1. The aging vessel is filled with 400 mL of the drilling fluid, and the drilling fluid is a common high-solid drilling fluid, which includes 10% bentonite+0.2% sodium hydroxide+0.1% xanthan gum. 8 g of sodium montmorillonite 500 is loaded into the nylon filter cloth 200.
S2. Two ends of the nylon filter cloth 200 are fixed using the first fixing frame 300 and the second fixing frame 400.
S3. The second fixing frame 400 is fixed to the inner wall of the bottom portion of the aging vessel, and the first fixing frame 300 is fixed to the inner wall of the top portion of the aging vessel.
S4. The temperature of a roller oven is raised to 230° C., and the aging vessel is placed inside the roller oven to perform hot rolling for 48 hours. The rotational speed of the roller oven is 50 r/min.
S5. After the hot rolling is completed, the aging vessel is cooled and opened. The nylon filter cloth 200 is taken out, and the sodium montmorillonite 500 is collected and placed into a centrifuge for centrifugation at 10000 r/min and 25° C. for 20 minutes.
S6. After the centrifugation, the supernatant liquid is poured out and the centrifuged sodium montmorillonite 500 is collected.
S7. The basal spacing of the centrifuged sodium montmorillonite 500 is determined by XRD under the following conditions: the diffraction wavelength λ=0.154056 nm, the operating voltage of 40 kV, the current of 30 mA, the scanning angle 2θ=3° to 40°. The inhibition performance of the drilling fluid is evaluated based on the basal spacing. The evaluation method is shown in Table 1 below.

TABLE 1

| | | Surface Hydration |
|---|---|---|
| | | Inhibition |
| Basal Spacing | Hydration Layers | Evaluation |
| Less than | Dry montmorillonite | No hydration |
| 1.00 nm | | |
| 1.00 nm to | Montmorillonite with | Good inhibition |
| 1.30 nm | 1 hydration layer | |
| 1.30 nm to | Montmorillonite with | Relatively good |
| 1.50 nm | 2 hydration layers | inhibition |
| 1.50 nm to | Montmorillonite with | Relatively poor |
| 1.80 nm | 3 hydration layers | inhibition |
| 1.80 nm to | Montmorillonite with | Poor inhibition |
| 2.00 nm | 4 hydration layers | |
| Greater than | Montmorillonite with | No inhibition |
| 2.00 nm | 5 or more hydration layers | |

In this embodiment, the basal spacing of the sodium montmorillonite 500 determined by XRD is 2.01 nm. Referring to Table 1, the surface hydration inhibition of the common high-solid drilling fluid is evaluated as having no inhibition.

### Comparative Example 5

This comparative example is basically the same as the comparative example 1, except that the drilling fluid is the common high-clay drilling fluid. After the test, according to Formula 1, the Sᵣ of the common high-clay drilling fluid is 55%, indicating poor inhibition or no inhibition. In the absence of a definitive evaluation criterion, the comparative example 5 could only roughly infer the inhibition strength from the Sᵣ value, concluding it was weak or absent—a finding consistent with the result of the embodiment 4. This consistency further validates the feasibility and accuracy of the method employed in the embodiment 4. However, since the disclosure establishes a clear benchmark for inhibition evaluation, it allows for a definitive conclusion that the drilling fluid system has no inhibition, unlike the uncertain conclusion in the comparative example 5. Therefore, the method of the disclosure is more accurate and reliable, thereby providing a clear standard for field personnel to accurately determine inhibition effectiveness and optimize inhibitor selection and dosage based on specific conditions.

The above described embodiments are merely part of the embodiments of the disclosure, not all of them. The detailed description of the embodiments of the disclosure is not intended to limit the scope of the claimed disclosure, but merely represents selected embodiments of the disclosure. Based on the embodiments of the disclosure, all other embodiments obtained by those of ordinary skill in the art without creative efforts shall fall within the protection scope of the disclosure.

What is claimed is:

1. A device for testing surface hydration inhibition of drilling fluid, comprising:
a reaction vessel;
a nylon filter cloth, disposed in the reaction vessel;
a first fixing frame, disposed in the reaction vessel; and
a second fixing frame, disposed in the reaction vessel;
wherein an end of the nylon filter cloth is detachably connected to the first fixing frame, and another end of the nylon filter cloth is detachably connected to the second fixing frame; and the first fixing frame is detachably connected to an inner wall of a top portion of the reaction vessel, and the second fixing frame is detachably connected to an inner wall of a bottom portion of the reaction vessel.

2. The device according to claim 1, wherein each of the first fixing frame and the second fixing frame is embedded with a first magnetic block, each of the inner wall of the top portion and the inner wall of the bottom portion of the reaction vessel is embedded with a second magnetic block, and the first magnetic block and the second magnetic block have opposite magnetic poles.

3. The device according to claim 1, wherein the reaction vessel is an aging vessel.

4. The device according to claim 1, wherein the nylon filter cloth has a mesh size greater than 0.1 micrometers.

5. A method for testing surface hydration inhibition of drilling fluid, based on the device according to claim 3, comprising the following steps:
S1, filling the aging vessel with 400 mL of the drilling fluid, and loading sodium montmorillonite with a purity greater than 99% into the nylon filter cloth;
S2, fixing two ends of the nylon filter cloth using the first fixing frame and the second fixing frame;
S3, fixing the second fixing frame to the inner wall of the bottom portion of the aging vessel, and fixing the first fixing frame to the inner wall of the top portion of the aging vessel;
S4, raising temperature of a roller oven to a specified temperature, and placing the aging vessel inside the roller oven to perform hot rolling;
S5, after the hot rolling is completed, cooling and opening the aging vessel, taking out the nylon filter cloth, taking out the sodium montmorillonite, and placing the sodium montmorillonite into a centrifuge for centrifugation;
S6, after the centrifugation, pouring out supernatant liquid and collecting centrifuged sodium montmorillonite; and
S7, determining basal spacing of the centrifuged sodium montmorillonite by using X-ray diffraction (XRD), and evaluating inhibition performance of the drilling fluid based on the basal spacing.

6. The method according to claim 5, wherein in S4, the specified temperature is in a range of 25° C. to 230° C., time for the hot rolling is a range of 12 hours to 48 hours, and a rotational speed of the roller oven is 50 r/min.

7. The method according to claim 5, wherein in S4, the specified temperature is in a range of 100° C. to 200° C.

8. The method according to claim 5, wherein in S5, the centrifugation comprises centrifugation with a rotational speed of 10000 r/min at 25° C. for 20 minutes.

9. The method according to claim 5, wherein in S7, parameters for the XRD comprise a diffraction wavelength λ of 0.154056 nm, an operating voltage of 40 kV, a current of 30 mA, and a scanning angle of 2θ from 3° to 40°.

10. The method according to claim 5, wherein in S7, criteria for evaluating the inhibition performance of the drilling fluid comprises:
when the basal spacing is less than 1.00 nm and the centrifuged sodium montmorillonite contains zero hydration layers, the surface hydration inhibition is evaluated as no hydration;
when the basal spacing is in a range of 1.00 nm to 1.30 nm and the centrifuged sodium montmorillonite contains one hydration layer, the surface hydration inhibition is evaluated as good inhibition;
when the basal spacing is in a range of 1.30 nm to 1.50 nm and the centrifuged sodium montmorillonite contains two hydration layers, the surface hydration inhibition is evaluated as relatively good inhibition;
when the basal spacing is in a range of 1.50 nm to 1.80 nm and the centrifuged sodium montmorillonite contains three hydration layers, the surface hydration inhibition is evaluated as relatively poor inhibition;
when the basal spacing is in a range of 1.80 nm to 2.00 nm and the centrifuged sodium montmorillonite contains four hydration layers, the surface hydration inhibition is evaluated as poor inhibition; or
when the basal spacing is greater than 2.00 nm and the centrifuged sodium montmorillonite contains five or more hydration layers, the surface hydration inhibition is evaluated as no inhibition.
